(19) United States
(12) Patent Application Publication
(10) Pub. No.: US 2021/0210183 A1
(43) Pub. Date: Jul. 8, 2021

(54) Semantic Graph Textual Coding

(71) Applicant: 3M INNOVATIVE PROPERTIES COMPANY, St. Paul, MN (US)

(72) Inventors: Jörg M. Niggemann, München (DE); Michael Owsijewitsch, Konstanz (DE); Hans-Jörg D. Schumann, Kreuzlingen (CH); Hans Rudolf Straub, Kreuzlingen (CH); Jeremy R. Kornbluth, Cheverly, MD (US); Gordon E. Johnson, Bethesda, MD (US)

(21) Appl. No.: 17/250,143

(22) PCT Filed: Jun. 26, 2019
(86) PCT No.: PCT/IB2019/055418

§ 371 Date: Dec. 7, 2020

Related U.S. Application Data
(60) Provisional application No. 62/692,048, filed on Jun. 29, 2018.

(51) Int. Cl. G16H 10/60; G06F 16/22; G16H 15/00

(52) U.S. Cl. CPC G16H 10/60; G16H 15/00; G06F 16/2264

(57) ABSTRACT

Various embodiments herein each include at least one of systems, methods, software, and data structures for semantic graph textual coding. While some embodiments are applicable to coding text of medical records for billing purposes, other embodiments are applicable to coding of any text, regardless to any number of different coding schemes, whether the coding scheme is a defined standard or an ad hoc code for a particular project. One method embodiment includes processing text of a new record to generate at least one concept molecule that represents semantic meaning of the text of the new record and comparing each of the at least one concept molecules to a set of target molecules to identify at least one closest matching target molecule to each of the at least one concept molecules. The method may then store a representation of a closest matching target molecule in association with the new record.

### BACKGROUND INFORMATION

Document coding is generally a process of mapping topics included in a document to a code of a code-set. The topics in different scenarios may simply be words but may also, or instead, be the real interest is in the semantic meanings of one or more words, sentences, and paragraphs within a document which is its semantics, consisting not of words but of concepts. The code-set to which a document is mapped may be unique to an organization or purpose but may instead be a standardized code-set as set by an industry organization, a government entity, a company, or as may be needed to integrate code-set data with a particular computer system or computing environment. Regardless, document coding is performed for many reasons such as organizing, indexing, inventorying, billing, and the like. The documents may be of different types for these purposes, such as legal documents which may include evidentiary documents, medical records of procedures and services provided, academic and technical articles and papers, and others.

Initially, document coding was performed manually. There has been an ongoing effort for electronically processing documents for automatic coding. These efforts have progressed but are generally rule-driven. Such rules often provide one-to-one or many-to-one mapping of words or a semantic meaning to one code. These rules are typically inflexible, difficult to define and update, and generally expensive to maintain due to hard-coding within computer programs or components thereof and the computer code and complexity of the rules generally being inaccessible to non-expert computer-coding employees.

### SUMMARY

Various embodiments herein each include at least one of systems, methods, software, and data structures for semantic graph textual coding. While some embodiments are applicable to coding text of medical records for billing purposes, other embodiments are applicable to coding of any text, regardless to any number of different coding schemes, whether the coding scheme is a defined standard or an ad hoc code for a particular project. One method embodiment includes processing text of a new record to generate at least one concept molecule that represents semantic meaning of the text of the new record and comparing each of the at least one concept molecules to a set of target molecules to identify at least one closest matching target molecule to each of the at least one concept molecules. The method may then store a representation of a closest matching target molecule in association with the new record.

Another method embodiment includes storing a set of target molecules, each target molecule of the set of target molecules representative of a respective code of a defined coding system. This method may then process text of a new record to generate at least one concept molecule that represents semantic meaning of the text of the new record. A comparing of each of the at least one concept molecules to the set of target molecules is then performed to identify at least one closest matching target molecule to each of the at least one concept molecules. A data representation of an identified closest matching target molecule may then be stored in association with the new record when there is only one closest matching target molecule to a respective concept molecule. In some embodiments, when more than one target molecule is identified, the method includes requesting user input with regard to each concept molecule for which more than one target molecule is identified.

A further embodiment, in the form of a system, includes a data processing device having at least one hardware processor and a natural language processor. The natural language processor is executable by the at least one hardware processor to process received input text and output at least one molecule data structure that represents a semantic meaning of the received input text. The system further includes at least one memory device that stores a set of target molecules, each target molecule of the set of target molecules representative of a respective code of a defined coding system. The at least one memory device further stores instructions executable by the at least one hardware processor to perform data processing activities. The data processing activities may include receiving input text of a new record and processing the received input text with the natural language processor to obtain at least one concept molecule data structure that represents semantic meaning of the text of the new record. The data processing activities also include comparing each of the at least one concept molecule to the set of target molecules to identify at least one closest matching target molecule to each of the at least one concept molecules. A data representation of an identified closest matching target molecule in association with the new record may then be stored on the at least one memory device when there is only one closest matching target molecule to a respective concept molecule.

### BRIEF DESCRIPTION OF THE DRAWINGS

FIG. 1 is a block flow diagram of a method, according to an example embodiment.

FIG. 2 illustrates semantic graphs, according to an example embodiment.

FIG. 3 is block flow diagram of a method, according to an example embodiment.

FIG. 4 is a logical block diagram of a system architecture, according to an example embodiment.

FIG. 5 is an architectural diagram of a system, according to an example embodiment.

FIG. 6 is block flow diagram of a method, according to an example embodiment.

FIG. 7 is a block diagram of a computing device, according to an example embodiment.

### DETAILED DESCRIPTION

Various embodiments herein each include at least one of systems, methods, software, and data structures for semantic graph textual coding. While some embodiments are applicable to coding text of medical records for billing purposes, other embodiments and the contributions herein are generally applicable to coding of virtually any text, regardless of source of the text or purpose, to any number of different coding schemes, whether the coding scheme is a defined standard or an ad hoc code for a particular project.

These various embodiments advance beyond just rules-based coding, instead also performing semantic graph textual coding. Textual coding refers to coding of text included in documents. When coding of a document or coding of text are referred to herein, the terms are generally interchangeable unless stated otherwise. Some embodiments, at a high-level, include processing of a coding scheme, including natural language processing, to build a set of target semantic graphs representing semantic meanings of codes to which documents are to be assigned. In some embodiments, text of a document to be coded is subjected to the same processing as the coding scheme to build one or more semantic graphs representing semantic meanings of the document text to be coded. Subsequently, the one or more semantic graphs representing semantic meanings of the document text to be coded are each compared to the target semantic graphs to identify matches thereto, or at least closest matches. The codes of the closest matching target semantic graphs are then associated with respective semantic graphs of the document text and data representing those associations is output to another process or stored.

Note that some coding schemes are equally applicable across each of a plurality of languages, such as English, German, French, Chinese, Japanese, and other languages. In such instances, the structure of a semantic graph of a coding scheme is the same, but there may be an instance of the structure tailored to the vernacular of the particular language to enable coding of input text for more than one language.

These and other embodiments are described in greater detail below with reference to the figures.

In the following detailed description, reference is made to the accompanying drawings that form a part hereof, and in which is shown by way of illustration specific embodiments in which the inventive subject matter may be practiced. These embodiments are described in sufficient detail to enable those skilled in the art to practice them, and it is to be understood that other embodiments may be utilized and that structural, logical, and electrical changes may be made without departing from the scope of the inventive subject matter. Such embodiments of the inventive subject matter may be referred to, individually and/or collectively, herein by the term “invention” merely for convenience and without intending to voluntarily limit the scope of this application to any single invention or inventive concept if more than one is in fact disclosed.

The following description is, therefore, not to be taken in a limited sense, and the scope of the inventive subject matter is defined by the appended claims.

The functions or algorithms described herein are implemented in hardware, software or a combination of software and hardware in one embodiment. The software comprises computer executable instructions stored on computer readable media such as memory or other type of storage devices. Further, described functions may correspond to modules, which may be software, hardware, firmware, or any combination thereof. Multiple functions are performed in one or more modules as desired, and the embodiments described are merely examples. The software is executed on a digital signal processor, ASIC, microprocessor, or other type of processor operating on a system, such as a personal computer, server, a router, or other device capable of processing data including network interconnection devices.

Some embodiments implement the functions in two or more specific interconnected hardware modules or devices with related control and data signals communicated between and through the modules, or as portions of an application-specific integrated circuit. Thus, the exemplary process flow is applicable to software, firmware, and hardware implementations.

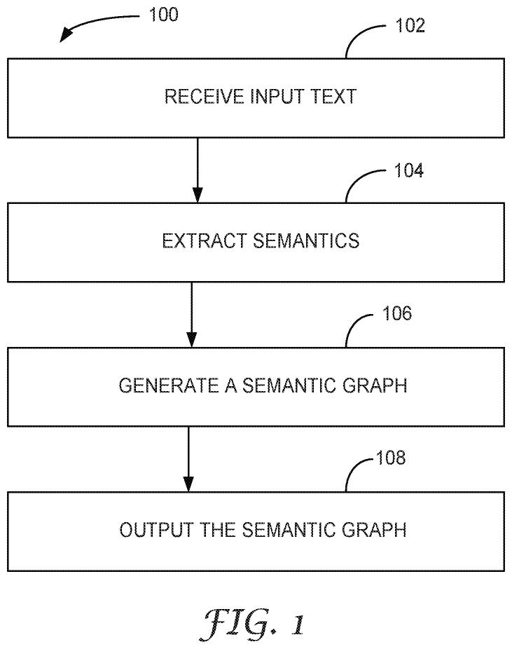

FIG. 1 is a block flow diagram of a method 100, according to an example embodiment. The method of claim 1 is an example of a computer implemented method that may be executed to build data representations of semantic graphs of text to be coded. The purpose for coding text according to the method 100 may be to build target semantic graphs to which new text is to be coded and building semantic graphs of next text to be coded. As target semantic graphs and semantic graphs of new text are built according to the same processing, the output semantic graphs are aligned structurally for purposes of later matching.

The method 100 includes receiving 102 input text and extracting 104 semantics therefrom. The method 100 then generates 106 one or more semantic 106 graphs for extracted 104 semantics and the one or more generated 106 semantic graphs are output 108, such as to a calling process or as data stored to a data storage device or a memory device.

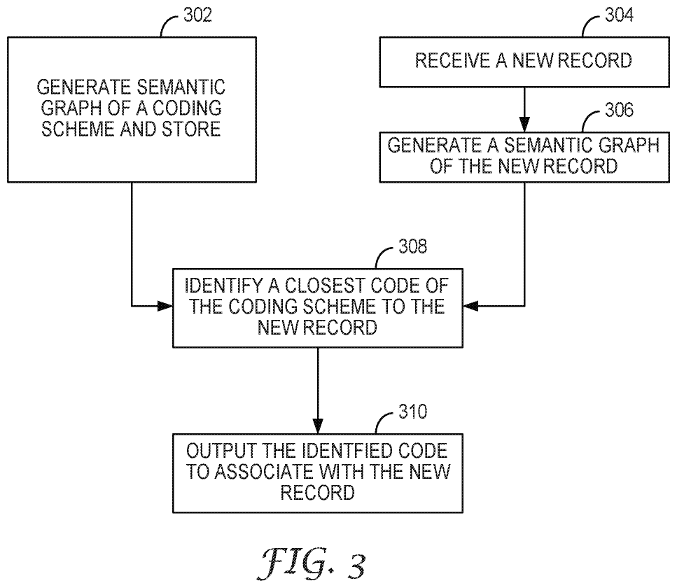

In some embodiments, the received 102 input text may be text of a coding scheme. In such embodiments, the semantic graph to be output 108 is a target semantic graph to which newly received 102 text is to be associated when subsequent coding is performed, such as according to the method 300 of FIG. 3. In some further embodiments, the received 102 input text may be text of a new record or document to be coded.

In some specific embodiments, received 102 input text may be a coding scheme defined for a particular purpose, such as by a governmental body, a consortium, a standard setting group, and the like. Some such coding schemes may be for medical billing, which may include facility and professional reimbursement fact coding elements. The facility reimbursement facts and the professional reimbursement facts may be any information related to reimbursement for the services performed and equipment used during a patient medical encounter. These reimbursement facts may include, but are not limited to, medical billing codes. Examples of such medical billing codes include codes associated with the International Classification of Diseases (ICD) codes (versions 9 and 10), Current Procedural Technology (CPT) codes, a Healthcare Common Procedural Coding System codes (HCPCS), and Physician Quality Reporting System (PQRS) codes. In some examples, the codes associated with facility reimbursement include ICD codes, CPT codes, and HCPCS codes. Generally, these reimbursement facts are related to the services and equipment provided by the facility where the patient encounter occurred. Codes associated with professional reimbursement may include ICD codes, CPT codes, and PQRS codes. Generally, these reimbursement facts are related to the services and equipment provided by the attending medical professional. In other examples, the facility and professional reimbursement facts may include any medical billing codes. Thus, in different instances when the method 100 is executed, the received 102 input text may be a coding scheme or text to be coded according to the coding scheme.

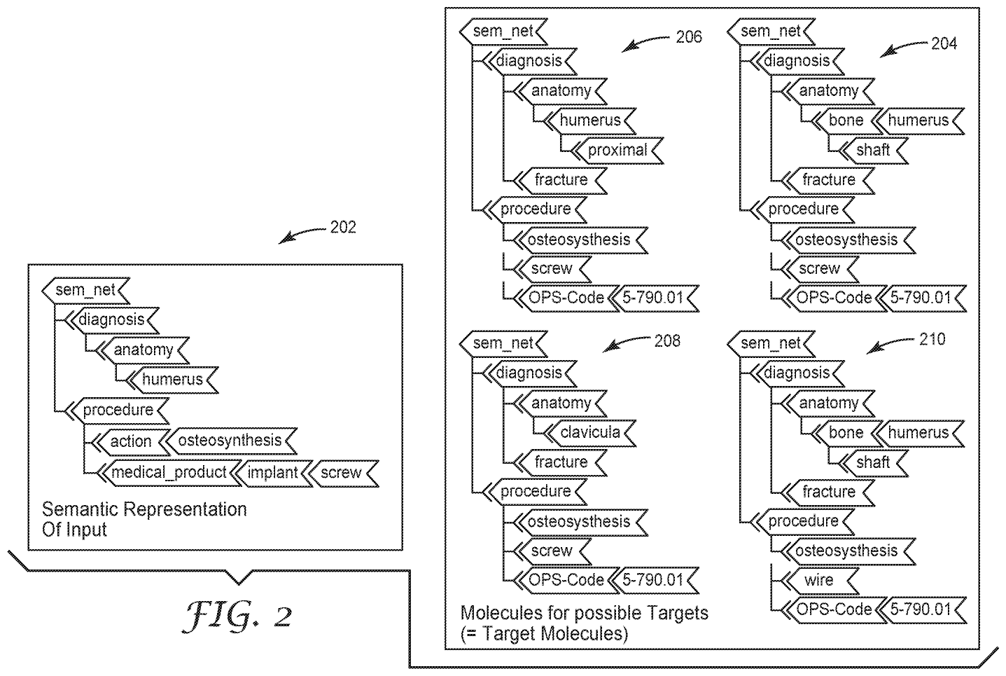

The extraction 104 of semantics, in some embodiments, is performed through natural language processing, the findings of which are utilized to generate 106 a semantic graph. The natural language processing is performed to find meaning from words. In some embodiments, a meaning is generally referred to as a concept. Such embodiments distinguish between concept types, such as atomic (single, simple and indivisible) and molecular (composite) concepts. The atomic concepts (concept atoms) are building blocks of the composite concepts (concept molecules). Concept molecules are represented in semantic graphs as data structures built from atoms that are arranged considering the semantic relations between them. The arrangement distinguishes between hierarchic and attributive relations. In the graphical representation of a molecule, hierarchic relations are shown horizontally and attributive relations vertically. The hierarchic relation is always between a concept and its subconcepts, the attributive one between a concept and its attributes. The concept molecule is thus a well-structured composite of atoms linked together with clearly distinguishable hierarchic and attributive relations. The resulting structure is in detail described by H. R. Straub in “Das Interpretierende System” (2001). Thus, a semantic graph includes at least a concept molecule with at least an atom but may also include one or more other concept molecules that each include one or more atoms. Thus, a conceptual semantic graph may be referred to as a at least one concept molecule built from at least one atoms. In some embodiments, a code of a coding scheme to which a concept molecule has been associated may be included as an atom of the concept molecule. Examples of atoms, concepts, and concept molecule data structures are included in FIG. 2.

FIG. 2 illustrates semantic graphs 202, 204, 206, 208, 210 according to an example embodiment. The semantic graph 202 on the left is an example of a semantic graph built from text received 102 for coding, such as a textual description of a medical procedure. The semantic graphs 204, 206, 208, 210 on the right are built from portions of a defined coding scheme, such as a medical billing coding scheme. As is readily visible in these semantic graphs 202, 204, 206, 208, 210 is that they are quite similar and readily understandable, even to someone without in-depth familiarity with medical terminology, as representing a medical procedure involving a screw or wire in a bone.

With regard to atoms as discussed above, each individual arrow element is an atom, such as “bone” and “humerus.” However, bone and humerus combined form a composite (molecular) concept as a humerus is a bone. Concepts can extend in other dimensions as well, such as to provide more specific detail with regard to a concept or to provide included or implied detail already present. For example, the atom “shaft” provides more specific location detail with regard to the “bone humerus” concept. Similar with included or implied details, the “bone humerus” concept is inclusive of the “anatomy” and “diagnosis” atoms, which they themselves are also concepts. The atoms of the semantic graphs 202, 204 thereby for the concepts represented thereby and the concepts together form the concept molecules of the respective semantic graphs 202, 204.

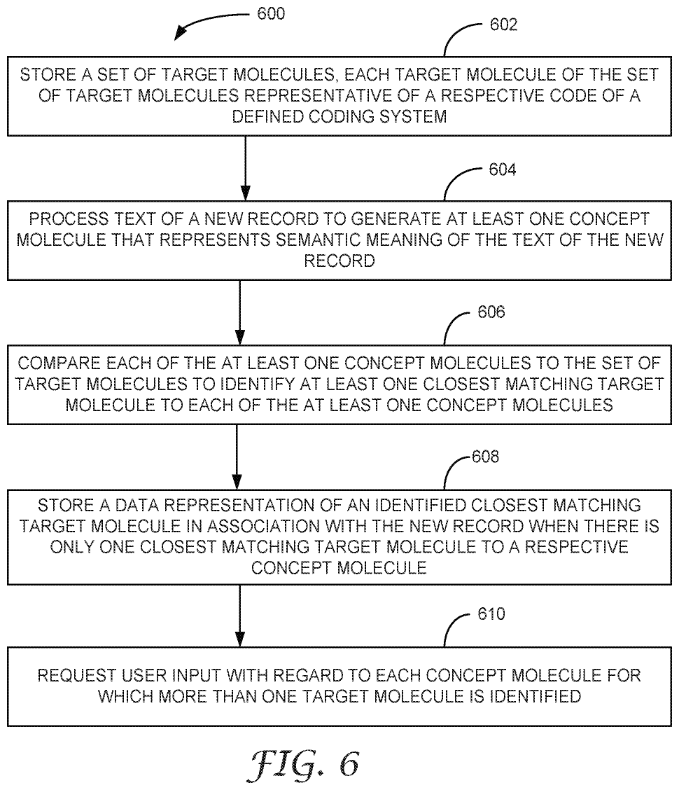

Of further note with regard to the righthand semantic graph 204 is that the semantic graph 204 has been coded as indicated by the bottom-most illustrated atoms “OPS-Code” and “5-790.02.” Subsequently, when the left-hand semantic graph 202 is processed for coding, such as by the method 300 of FIG. 3 or in relation to the method 600 of FIG. 6, the closest matching semantic graph to the left-hand semantic graph 202 is the right-hand semantic graph 204.

Looking more closely at the other semantic graphs 206, 208, 210 in comparison to the semantic graph 202 that was generated from input text, mismatches are readily visible. For example, the semantic graphs 206 and 210 do not involve the humerus shaft and the semantic graphs 208 involves a wire procedure.

FIG. 3 is block flow diagram of a method 300, according to an example embodiment. The method 300 is an example of a computerized method that maybe performed on one or more computing devices to code textual documents. The method 300 includes generating 302 a semantic graph of a coding scheme and storing it for later use to identify 308 a closest code of the coding scheme to a newly received record 304. Note that in some embodiments, a single, generated 302 semantic graph is representative of an entire coding scheme. However, in other embodiments, a semantic graph may be generated 302 for each of a plurality of codes included in a coding scheme.

As such, the method 300 also includes receiving 304 a new record. A new record that is received 304 may include a textual medical record of a patient's visit with a doctor, a procedure received by a patient, equipment used during a visit or treatment, facilities utilized, and combinations thereof. However, a new record may alternatively be other textual information such as factual or opinion documents in a legal matter, technical writings, government documents, and other textual documents that are to be coded. Accordingly, the generated 302 semantic graph of the coding scheme is generated 302 from a coding scheme generally topically tailored to, or inclusive of, the subject of the received 304 record.

The newly received 304 record is then processed to generate 306 a semantic graph representative thereof. Generating 306 a semantic graph from the newly received 304 record, in some embodiments, includes generating a semantic graph for each fact of interest to the coding scheme, such as each diagnosis, procedure, and the like in a medical context. In some embodiments, the generated 302, 306 semantic graphs are generated 302, 306 by the same process, either actually or logically. In some embodiments, this process by which the semantic graphs are generated 302, 306 may be a process according to the method 100 of FIG. 1.

The method 300 then proceeds in some embodiments by identifying 308 a closest code of the coding scheme to the newly received 304 record. The closest code may be identified 308 in some such embodiments by comparing the generated 306 semantic graph of the new record to the stored semantic graph generated 302 from the coding scheme. The closest code in some embodiments is identified based on full or partial matching of their semantic graphs, such as matching a semantic graph or concept molecule as described with regard to FIG. 2 above with a target semantic graph, as described above. This matching may also be referred to as a matching of a concept molecule generated from the received 304 new record with a target molecule of a plurality of target molecules generated 302 from the coding scheme.

The matching may be an exact matching or a relative matching assisted by a scoring scheme, closest neighbor algorithm, and the like. For example, the semantic graphs 202, 204 of FIG. 2, although generated by the same process, are slightly different. Such differences typically occur if the 304 text—and in consequence the 306 semantic graph—are more detailed than the 302 semantic graph which represents the meaning of the code. If there is no 302 semantic graph that represents the full details of 304 and in consequence 306, the assignment and output of a more general 308 code is fully correct. The match algorithm considers this possibility and despite their differences, the two graphs 302 and 306 are deemed to be matching in such cases.

The method 300, after identifying 308 a closest code of the coding scheme to the new record, may then output 310 the identified 308 code to associate with the new record. Outputting 310 the identified 308 code may include storing the code in association with the new record, augmenting a data structure of the new record with the identified 308 code as the new record flows through a data processing pipeline that includes the method 300, returning the identified 308 code to a process that called the method 300 to be performed, and the like.

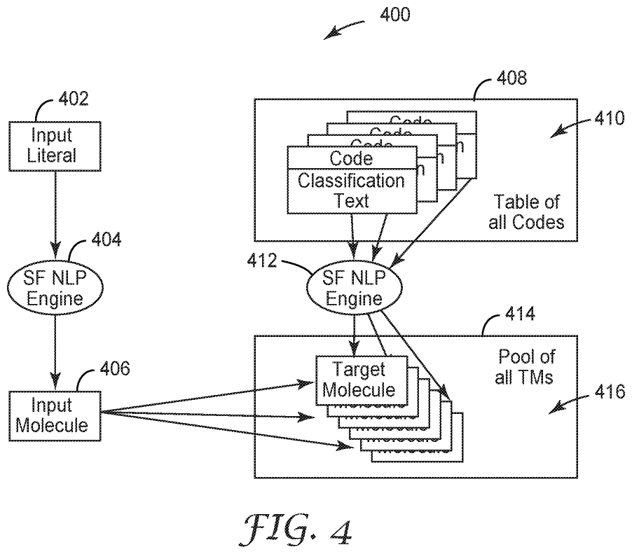

FIG. 4 is a logical block diagram of a system 400 architecture, according to an example embodiment. The system 400 includes at least one natural language processing (NLP) engine 404, 412 that are deployed on computing resources to process input text, such as an input literal 402 (e.g., a record to be coded) or one or more documents 408 defining a coding scheme including textual descriptions of each of a plurality of codes 410. The NLP 404, 412 outputs at least one semantic graph for each input document 402, 408. The output semantic graph may be an input molecule 406 that is to be coded according to a pool of target molecules 414 that include a target molecule 416 for each of the plurality of codes 410. The coding of an input molecule 406 to one or more of the target molecules 416, in some embodiments, is performed according to the method 300 of FIG. 3.

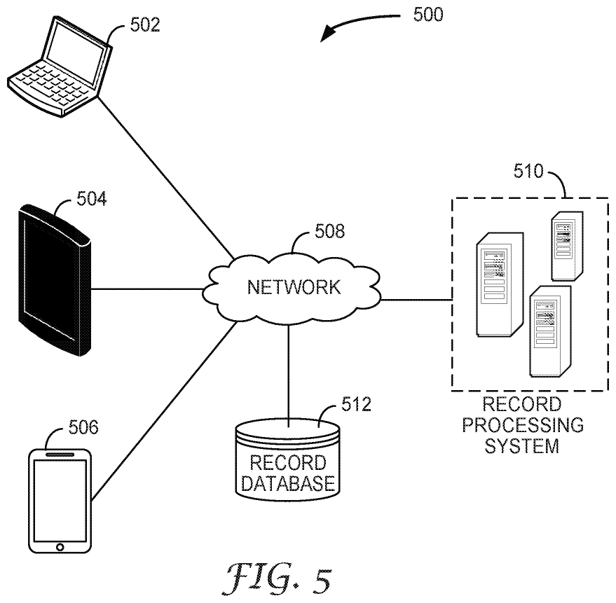

FIG. 5 is an architectural diagram of a system 500, according to an example embodiment. The system 500 includes one or more devices 502, 504, 506 through which users may interact with the system 500, such as to input text to generate records that are eventually coded, to initiate semantic graphing of a coding scheme, to review coding results, to edit, confirm, or modify automatic coding of a document, and the like. The one or more device may include one or more of each of a personal computer 502, a tablet 504, a smartphone 506, a client virtual machine, and other computing devices. The system 500 also includes a network 508 that interconnects the devices 502, 504, 506, a record processing system 510, and a record database 512.

The record processing system 510 is a system that includes software to perform data processing activities of and related to semantic graph textual coding. For example, the record processing system 510 may perform all or a portion of one or both of the methods 100 and 300 of FIG. 1 and FIG. 3, respectively. The record processing system 510 may be one or more physical servers with the software deployed thereon. In other embodiments, the record processing system 510 may be deployed on one or more virtual machines. However, in some other embodiments, record processing system 510 software may be deployed directly on one of the devices 502, 504, 506.

The system 500 may also include a record database 512 to which textual records, data of textual coding, molecules, and other data may be stored or updated. The record database 512 may be a relational database, one or more flat files, or store data according to another scheme.

FIG. 6 is block flow diagram of a method 600, according to an example embodiment. The method 600 is an example of a semantic graph textual coding method that may be performed by one or more computing devices.

The method 600 includes storing 602 a set of target molecules. Each target molecule of the set of target molecules is typically representative of a respective code of a defined coding system. The method 600 also includes processing 604 text of a new record to generate at least one concept molecule that represents semantic meaning of the text of the new record and subsequently comparing 606 each of the at least one concept molecules to the set of target molecules. The comparing 606 is performed to identify at least one closest matching target molecule to each of the at least one concept molecules. The method 600 may them store 608, on a data storage device, a data representation of an identified closest matching target molecule in association with the new record when there is only one closest matching target molecule to a respective concept molecule. However, the method 600 includes requesting 610 user input with regard to each input molecule for which more than one alternative target molecule is identified.

In some embodiments, the target molecules of the stored 602 set of target molecules is generated through textual processing. The textual processing of such embodiments may include processing text of the defined coding system according to a natural language processing scheme to generate the set of target molecules. Such embodiments then output the target molecules of the set of target molecules for storing on a data storage device.

In some other embodiments, the processing 604 of the text of the new record to generate the at least one concept molecule includes processing the new record according to the same natural language processing scheme such that the stored 602 data representation of the concept molecule has an identical representative structure to the target molecules of the set of target molecules. In semantic graphs, representative structures of each of the target molecules and each of the at least one concept molecules are multi-dimensional data structures of semantic relationships of text represented thereby (e.g., see FIG. 2). Such multi-dimensional data structures may include. In particular, an atom in the first direction provides specificity to the atom it is related to and the atoms added in the second direction provide specific characteristics grouped in one or more attributive types to it. The comparing 606 in such embodiments may include matching of elements—in both directions.

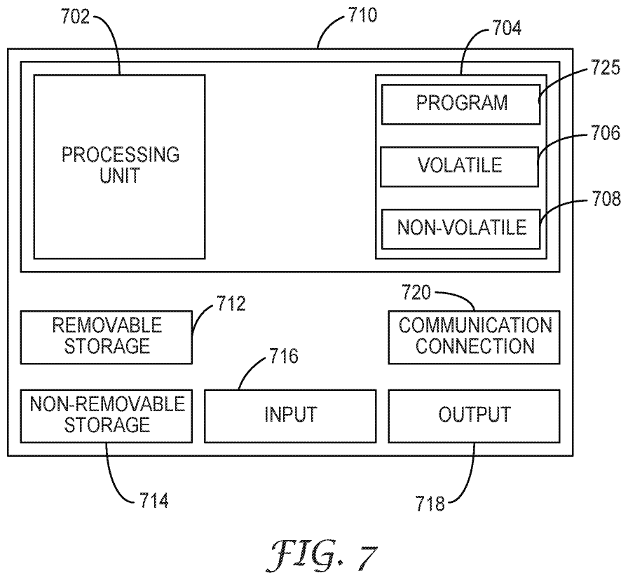

FIG. 7 is a block diagram of a computing device, according to an example embodiment. In one embodiment, multiple such computer systems are utilized in a distributed network to implement multiple components in a transaction-based environment. An object-oriented, service-oriented, or other architecture may be used to implement such functions and communicate between the multiple systems and components. One example computing device in the form of a computer 710, may include a processing unit 702, memory 704, removable storage 712, and non-removable storage 714. Although the example computing device is illustrated and described as computer 710, the computing device may be in different forms in different embodiments. For example, the computing device may instead be a smartphone, a tablet, smartwatch, or other computing device including the same or similar elements as illustrated and described with regard to FIG. 7. Devices such as smartphones, tablets, and smartwatches are generally collectively referred to as mobile devices. Further, although the various data storage elements are illustrated as part of the computer 710, the storage may also or alternatively include cloud-based storage accessible via a network, such as the Internet.

Returning to the computer 710, memory 704 may include volatile memory 706 and non-volatile memory 708. Computer 710 may include—or have access to a computing environment that includes a variety of computer-readable media, such as volatile memory 706 and non-volatile memory 708, removable storage 712 and non-removable storage 714. Computer storage includes random access memory (RAM), read only memory (ROM), erasable programmable read-only memory (EPROM) and electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 710 may include or have access to a computing environment that includes input 716, output 718, and a communication connection 720. The input 716 may include one or more of a touchscreen, touchpad, mouse, keyboard, camera, one or more device-specific buttons, one or more sensors integrated within or coupled via wired or wireless data connections to the computer 710, and other input devices. The computer 710 may operate in a networked environment using a communication connection 720 to connect to one or more remote computers, such as database servers, web servers, and other computing device. An example remote computer may include a personal computer (PC), server, router, network PC, a peer device or other common network node, or the like. The communication connection 720 may be a network interface device such as one or both of an Ethernet card and a wireless card or circuit that may be connected to a network. The network may include one or more of a Local Area Network (LAN), a Wide Area Network (WAN), the Internet, and other networks. In some embodiments, the communication connection 720 may also or alternatively include a transceiver device, such as a BLUETOOTH® device that enables the computer 710 to wirelessly receive data from and transmit data to other BLUETOOTH® devices.

Computer-readable instructions stored on a computer-readable medium are executable by the processing unit 702 of the computer 710. A hard drive (magnetic disk or solid state), CD-ROM, and RAM are some examples of articles including a non-transitory computer-readable medium. For example, various computer programs 725 or apps, such as one or more applications and modules implementing one or more of the methods illustrated and described herein or an app or application that executes on a mobile device or is accessible via a web browser, may be stored on a non-transitory computer-readable medium. For example, the computer programs 725 may include software of a natural language processing engine and software executable by the processing unit 702 to perform one or both of the methods 100, 300, and 600 of FIG. 1, FIG. 3, and FIG. 6, respectively.

Another system embodiments includes a computing device having at least one hardware processor and a natural language processor executable by the at least one hardware processor to process received input text and output at least one molecule data structure that represents a semantic meaning of the received input text. The computing device further includes at least one memory device storing a set of target molecules where each target molecule of the set of target molecules is representative of a respective code of a defined coding system. The at least one memory device also stores instructions executable by the at least one hardware processor to perform data processing activities.

The data processing activities may include receiving input text of a new record and processing the received input text of the new record. The processing of the received input text of the new record is performed in part with the natural language processor to obtain at least one concept molecule data structure that represents semantic meaning of the text of the new record. In some embodiments, the data processing activities further include matching concept molecules to target molecules. The matching, in some embodiments, includes comparing each of the at least one concept molecule to the set of target molecules to identify at least one closest matching target molecule to each of the at least one concept molecules. The matching may identify a perfect match, such as when the atoms of the target molecule are a subset of the atoms of the input molecule. Such a match generally means that all atoms of the target molecule are found in the input molecule. Atoms of the input molecule not found in the target molecule (regardless of the dimensions) do not contradict a positive match. The absent atoms are instead specifications in the input text which are unknown in the coding system and therefore not to be used for coding as they do not hinder a good match. However, challenges can occur when two target molecules partly match, each one of them specifying an information (atom) of the input molecule, which the other one does not. In such instances, additional information or input is requested or retrieved to determine which one of the two partly matching codes is to be preferred. Alternatively, such as when human input and other informational data sources are not available both codes may be output because both are “not wrong” or other methods may be utilized to identify a preferred code to output.

The data processing activities also include storing, on the at least one memory device, a data representation of an identified closest matching target molecule in association with the new record when there is only one closest matching target molecule to a respective concept molecule.

In some embodiments, the data processing activities further include requesting user input with regard to each concept molecule for which more than one target molecule is identified and receiving user input selecting a closest matching target molecule. A data representation of the user selected closest matching target molecule is then stored on the at least one memory device in association with the new record.

In some embodiments of this system, identifying at least one closest matching target molecule to each of the at least one concept molecules includes a scoring algorithm that assigns point values for attributive concepts matching between the concept molecule and a target molecule. Subsequent to the scoring, the closest match may be identified based on a score of one or more target molecules with a desired relative score.

It will be readily understood to those skilled in the art that various other changes in the details, material, and arrangements of the parts and method stages which have been described and illustrated in order to explain the nature of the inventive subject matter may be made without departing from the principles and scope of the inventive subject matter as expressed in the subjoined claims.

1. A method comprising:
storing a set of target molecules, each target molecule of the set of target molecules representative of a respective code of a defined coding system;
processing text of a new record to generate at least one concept molecule that represents semantic meaning of the text of the new record;
comparing each of the at least one concept molecules to the set of target molecules to identify at least one closest matching target molecule to each of the at least one concept molecules;
storing, on a data storage device, a data representation of an identified closest matching target molecule in association with the new record when there is only one closest matching target molecule to a respective concept molecule; and
requesting user input with regard to each concept molecule for which more than one target molecule is identified.

2. The method of claim 1, wherein the target molecules of the set of target molecules is generated through textual processing comprising:
processing text of the defined coding system according to a natural language processing scheme to generate the set of target molecules; and
outputting the target molecules of the set of target molecules for storing on the data storage device.

3. The method of claim 1, wherein the processing of the text of the new record to generate the at least one concept molecule includes processing the new record according to the same natural language processing scheme such that the data representation of the concept molecule has an identical representative structure to the target molecules of the set of target molecules.

4. The method of claim 3, wherein representative structures of each of the target molecules and each of the at least one concept molecules are multi-dimensional data structures of semantic relationships of text represented thereby.

5. The method of claim 4, wherein:
the multi-dimensional data structures include subconcepts added in one dimension and attributes of one or more attributive types added in another dimension.

6. The method of claim 1, wherein:
the defined coding system is a medical coding system; and
the new record is a textual representation of at least one of medical services, diagnoses, facilities, equipment, and procedures.

7. A system comprising:
at least one hardware processor;
a natural language processor executable by the at least one hardware processor to process received input text and output at least one molecule data structure that represents a semantic meaning of the received input text;
at least one memory device storing:
a set of target molecules, each target molecule of the set of target molecules representative of a respective code of a defined coding system;
instructions executable by the at least one hardware processor to perform data processing activities comprising:
receiving input text of a new record;
processing the received input text of the new record with the natural language processor to obtain at least one concept molecule data structure that represents semantic meaning of the text of the new record;
comparing each of the at least one concept molecule to the set of target molecules to identify at least one closest matching target molecule to each of the at least one concept molecules;
storing, on the at least one memory device, a data representation of an identified closest matching target molecule in association with the new record when there is only one closest matching target molecule to a respective concept molecule.

8. The system of claim 7, wherein the data processing activities further comprising:
requesting user input with regard to each concept molecule for which more than one target molecule is identified;
receiving user input selecting a closest matching target molecule from the more than one identified target molecules; and
storing, on the at least one memory device, the data representation of the user selected closest matching target molecule in association with the new record.

9. The system of claim 7, wherein the target molecules of the set of target molecules stored by the at least one memory device are generated by the natural language processor and have the same representative structure.

10. The system of claim 9, wherein:
the representative structure of each of the target molecules and each of the at least one concept molecules is a semantic graph of semantic relationships of text represented thereby; and
the semantic graph includes at least one atomic concept and when there are two or more atomic concepts, the atomic concepts are bound together by hierarchical relations in one direction and attributive relations in the other one.

11. The system of claim 10, wherein attributive relations originate from one atom in a vertical direction and are themselves structured according to the attributive qualities of the attributed atom.

12. The system of claim 10, wherein:
identifying at least one closest matching target molecule to each of the at least one concept molecules includes a scoring algorithm that assigns point values for associative and attributive concepts matching between the concept molecule and a target molecule; and
the closest match is identified based on a score of one or more target molecules with a desired relative score.

13. The system of claim 7, wherein each target molecule of the set of target molecules stored by the at least one memory device includes a code of the defined coding system.

14. The system of claim 13, wherein storing the data representation of the identified closest matching target molecule in association with the new record includes storing the code of the closest matching target molecule in association with the new record, the storing of the code indicating a coding of the new record for a purpose of the defined coding system.

15. The system of claim 14, wherein:
the new record is a textual representation of at least one of medical services and procedures rendered to a patient; and
the defined coding system is a medical services and procedures coding system.

16. A non-transitory computer readable medium, with instructions stored thereon that are executable by at least one hardware computer processor to perform data processing activities comprising:
processing text of a new record to generate at least one concept molecule that represents semantic meaning of the text of the new record;
comparing each of the at least one concept molecules to a set of target molecules to identify at least one closest matching target molecule to each of the at least one concept molecules;
requesting and receiving user input to identify a closest matching target molecule when the comparing identifies more than one closest matching target molecule;
storing, on a data storage device, a representation of the closest matching target molecule in association with the new record.

17. The non-transitory computer readable medium of claim 16, wherein the target molecules of the set of target molecules is generated through textual processing comprising:
processing text of a defined coding system according to a natural language processing scheme to generate the set of target molecules; and
outputting the target molecules of the set of target molecules for storing on the data storage device.

18. The non-transitory computer readable medium of claim 17, wherein the processing of the text of the new record to generate the at least one concept molecule includes processing the new record according to the same natural language processing scheme such that the data representation of the concept molecule has an identical representative structure to the target molecules of the set of target molecules.

19. The non-transitory computer readable medium of claim 18, wherein:
representative structures of each of the target molecules and each of the at least one concept molecules is a semantic graph of semantic relationships of text represented thereby; and
the semantic graph includes sub-concepts in one dimension and attributes in another dimension.
